(19) United States
(12) Patent Application Publication
(10) Pub. No.: US 2011/0066102 A1
(43) Pub. Date: Mar. 17, 2011

(54) Irrigation Device

(76) Inventor: Marsha Garcia, Jacksonville, FL (US)

(21) Appl. No.: 12/880,111

(22) Filed: Sep. 12, 2010

Related U.S. Application Data
(60) Provisional application No. 61/241,956, filed on Sep. 13, 2009.

(51) Int. Cl. A61M 3/02

(52) U.S. Cl. 604/36; 604/187; 604/28

(57) ABSTRACT

An irrigation device for irrigating a body cavity, such as an ear canal, is disclosed. The irrigation device comprises a hand-held pumping device and an irrigating tip. The hand-held pumping device includes a liquid reservoir and a pumping mechanism, which may be in the form of a spray bottle with a squeeze trigger. The outlet of the pumping mechanism is directly connected to the irrigating tip. The irrigating tip includes a connector and a short end or irrigation tube that is made of a flexible material and has inner and outer dimensions and a length suitable for insertion into the ear canal. When liquid is drawn from the reservoir and propelled through the irrigating tip, the irrigating tip oscillates as the fluid passes through it and into the ear canal. Kits including an irrigating device, irrigating tips, a basin, and a holder for the irrigating device are also disclosed.

### CROSS-REFERENCE TO RELATED APPLICATIONS

This application claims priority to U.S. Provisional Patent Application No. 61/241,956, filed Sep. 13, 2009. The contents of that application are incorporated by reference herein in their entirety.

### BACKGROUND OF THE INVENTION

1. Field of the Invention

The invention relates generally to the field of devices for irrigating and cleaning body cavities, and more particularly, to devices for cleaning and irrigating the ear canal.

2. Description of Related Art

Ear wax, also called cerumen, is secreted by the human ear canal and provides protection, cleaning, and lubrication for the structures within the ear. However, build-up of ear wax can also put pressure on the structures within the ear and can impede hearing. Several techniques are known for removing ear wax, including techniques using metal scraping tools (e.g., curettes and ear picks), and techniques using liquid-filled syringes, which are used to inject streams of liquid into the ear to clear the wax. Each of these methods has its disadvantages: using metal scraping tools, it is possible to damage the structures of the ear; using a syringe, control of the liquid stream may be awkward or difficult, and the stream will not be accurately aimed or caused to oscillate.

U.S. Pat. No. 5,833,675 to Teddy Garcia, which is incorporated by reference in its entirety, discloses an alternate solution. In the Garcia patent, a hand pump with a liquid reservoir is used to generate a pressurized stream of liquid, which is directed into a relatively long tube that is connected to a soft end tube of a smaller bore. The material of the end tube is flexible enough to oscillate when water is pumped through it. Although this device may function well in producing an oscillatory stream of fluid to the ear, the device itself may be difficult for a single person to manipulate and use.

### SUMMARY OF THE INVENTION

One aspect of the invention relates to an irrigation device for irrigating a body cavity, such as an ear canal. The irrigation device comprises a hand-held pumping device and an irrigating tip. The hand-held pumping device includes a liquid reservoir and a pumping mechanism, which may be in the form of a spray bottle with a squeeze trigger. The outlet of the pumping mechanism is directly connected to the irrigating tip. The irrigating tip includes a connector and a short end or irrigation tube that is made of a flexible material and has inner and outer dimensions and a length suitable for insertion into the ear canal. When liquid is drawn from the reservoir and propelled through the irrigating tip, the irrigating tip oscillates as the fluid passes through it and into the ear canal.

Other aspects, features, and advantages of the invention will be set forth in the description that follows.

### BRIEF DESCRIPTION OF THE DRAWING FIGURES

The invention will be described with respect to the following drawing figures, in which like reference numerals refer to like features throughout the figures, and in which:

FIG. 1 is a side elevational view of a portion of an irrigator according to one embodiment of the invention;

FIG. 2 is a top plan view of the irrigator of FIG. 1;

FIG. 3 is an exploded (disassembled) view of the outlet and irrigating tip of the irrigator;

FIG. 4 is a side elevational view of a portion of an irrigator according to another embodiment of the invention; and

FIG. 5 is a perspective view of an irrigation kit according to yet another embodiment of the invention.

### DETAILED DESCRIPTION

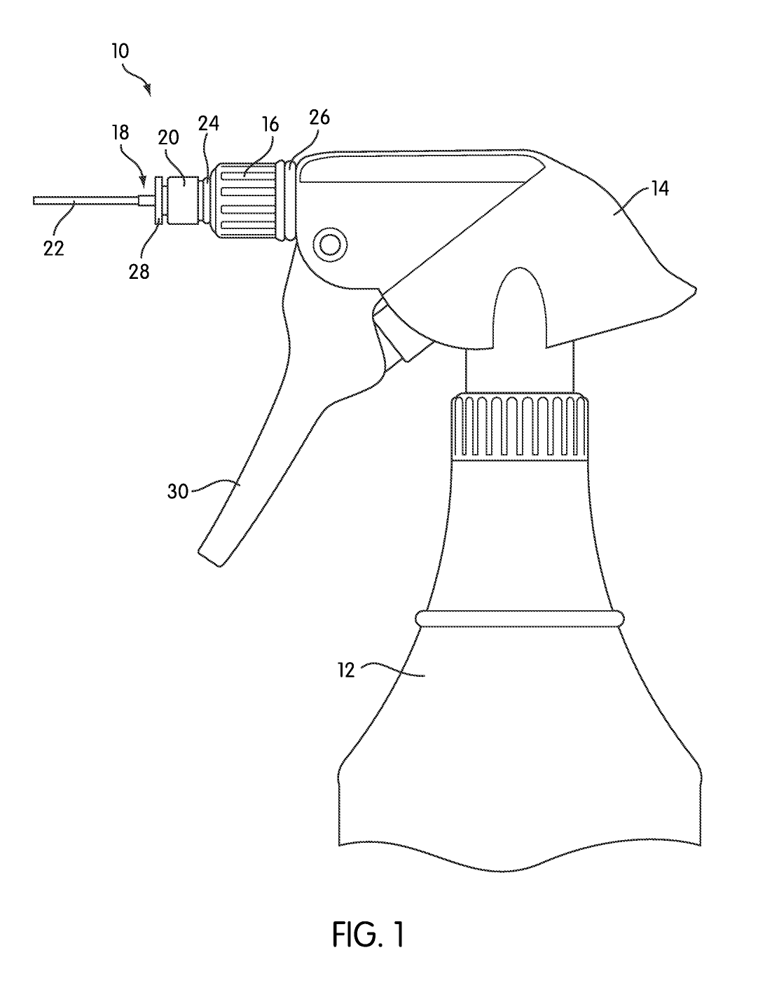

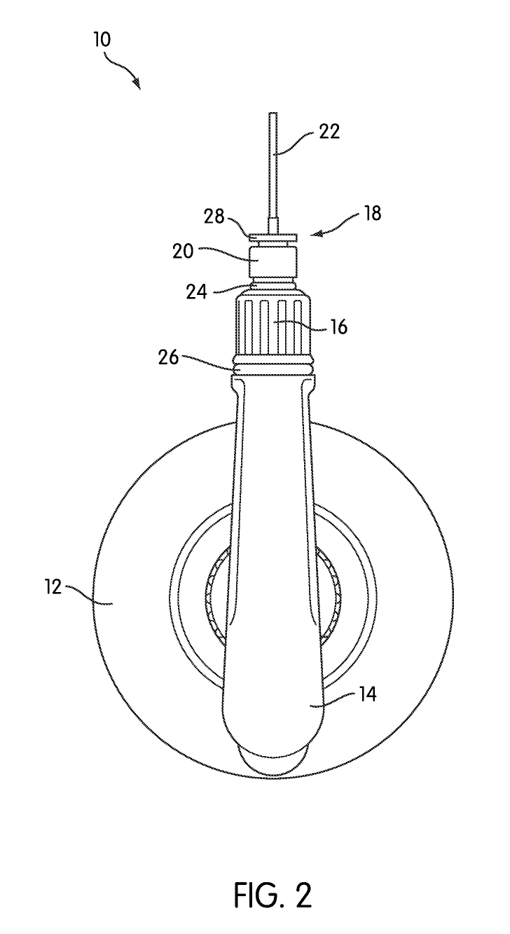

FIG. 1 and FIG. 2 are side elevational and top plan views of an irrigator, generally indicated at 10, according to one embodiment of the invention. The irrigator 10 comprises a liquid reservoir 12 connected to a mechanical pumping mechanism 14 that is coupled to the reservoir 12 and arranged such that when actuated, liquid is drawn from the reservoir 12 and expelled from the outlet 16 of the pumping mechanism 14. In the embodiment of FIGS. 1 and 2, the pumping mechanism 14 is a manual pump that has the form and attributes of a conventional spray bottle with a squeeze trigger 30, although pumping mechanisms having other forms and attributes may be used in other embodiments of the invention.

At the outlet 16 of the pumping mechanism 14, an irrigating tip 18 is connected. The irrigating tip 18 comprises a connector 28 and an end or irrigation tube 22 that in most embodiments is fixedly attached to the connector 28, such that the two components may be removed and replaced together, allowing the irrigator 10 to be used with multiple patients. The connector 28 connects on one end with the outlet 16 of the pumping mechanism 14 through an adapter 20 to place the end tube 22 in fluid communication with the pumping mechanism 14 and reservoir 12, so that liquid expelled from the outlet 16 is propelled through the end tube 22. The connection between the adapter 20 and the outlet 16 may, for example, be a threaded connection, and a first sealing structure 24, such as an O-ring, may be used to seal the connection between the two components 16, 20 and prevent leakage. If the pumping mechanism 14 is a conventional spray bottle, a second sealing structure 26, such as another, larger O-ring, may be used between the outlet 16 and the main body of the pumping mechanism 14 to maintain the outlet 16 in an open condition (if the outlet 16 is movable and has both open and closed positions) and to prevent leakage between the outlet 16 and the main body of the pumping mechanism 14.

The end tube 22 is generally comprised of a flexible tubing material of an outer diameter small enough to fit within the ear canal. The material of which the end tube is made would generally be a plastic or rubber, and may be flexible enough so that when liquid is expelled through the end tube 22, the end tube 22 oscillates as illustrated in U.S. Pat. No. 5,833,675. In general, any biocompatible tubing of appropriate size and compliance may be used. The end tube 22 may, for example, be made of polyvinyl chloride (PVC), while the connector 28 may be made of polypropylene. The end tube 22 may have a length on the order of about 0.5 inches to about 1 inch, and, for example, an outer diameter of ⅛ inch and an inner diameter of 1/16 inch.

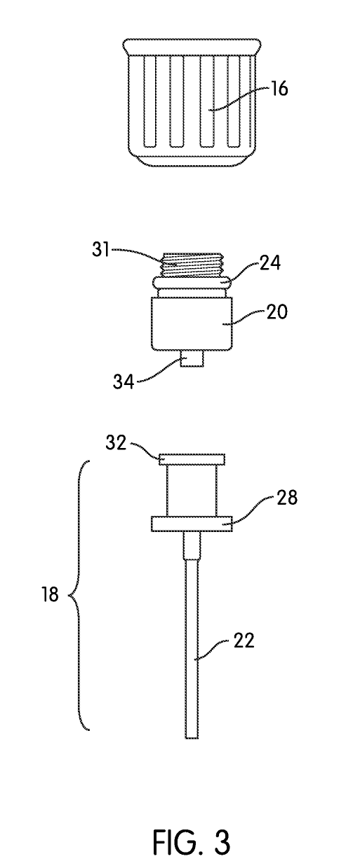

FIG. 3 is an exploded view of the outlet 16, the adapter 20, the connector 28, and a portion of the end tube 22, showing how the components fit together. The rearward portion 31 of the adapter 20 that faces the outlet 16 has male, exterior threads, carries the first sealing structure or O-ring 24, and makes a threaded connection with the outlet 16, which has complementary female threads. At its forward end 34, the adapter 20 forms a tubular portion of a smaller diameter that has threads or other appropriate connecting structure and is thus adapted to connect with the rearward portion 32 of the connector 28, which features an appropriately sized complementary threaded tubular connector.

The outlet 16 and adapter 20 are separable components in the illustrated embodiment, which is advantageous in that it allows the irrigating tip 18 to be attached easily to an existing pumping mechanism. However, in some embodiments, the outlet 16 and adapter 20 may be molded or otherwise formed as a single component. In yet other embodiments, the outlet itself may be formed so that it provides an attachment point suitable for the direct attachment of an irrigating tip 18.

As those of skill in the art will appreciate, although male threads are shown on some components and female threads on others, the sense of the threads may be reversed. Moreover, any form of connection may be used between the components, including, for example, luer-type connectors, especially between the irrigating tip 18 and the outlet 16 or adapter 20.

In the irrigator 10, there is no tubing or other flexible conduit between the outlet 16 of the pumping mechanism 14 and the irrigating tip 18. At least in part for that reason, the irrigator 10 can easily be operated by a single person using only one hand, with the end tube 22 inserted directly into the ear canal or other body cavity requiring irrigation and the rest of the irrigator 10 just outside and immediately proximate. The reservoir 12, pump mechanism 14 and other components may be particularly sized or adapted for one-handed usage in some embodiments.

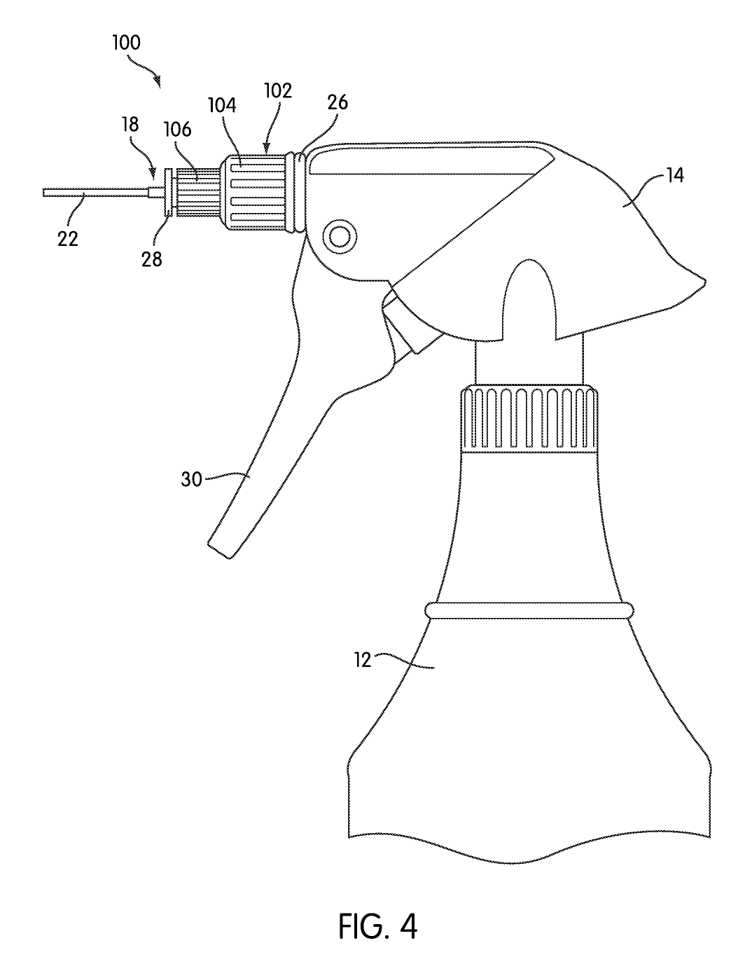

FIG. 4 is a side elevational view of an irrigator 100 according to another embodiment of the invention. The irrigator 100 has a reservoir 12 and a pump mechanism 14 similar to those of the irrigator 10, and other features not described here may also be assumed to be substantially similar to those of the irrigator 10. As was noted above, in some embodiments, the outlet 16 and adapter 20 may be made integral, as they are in the irrigator 100. Specifically, a unitary outlet member 102 in the irrigator 100 tapers from a pump-interfacing portion 104 of greater diameter to an irrigating tip interfacing portion 106 of somewhat lesser diameter, although not all embodiments need taper or change diameter. The unitary outlet member 102 may be formed in a single piece, e.g., using injection molding, and eliminates the need for a sealing member 24.

Interchangeable irrigating tips 18 would generally connect with the irrigating tip interfacing portion 106 in the same way as with the irrigator 10. Specifically, the inside diameter of the irrigating tip interfacing portion 106 may have threads or ridges that engage the connector 28 of the irrigating tip 18.

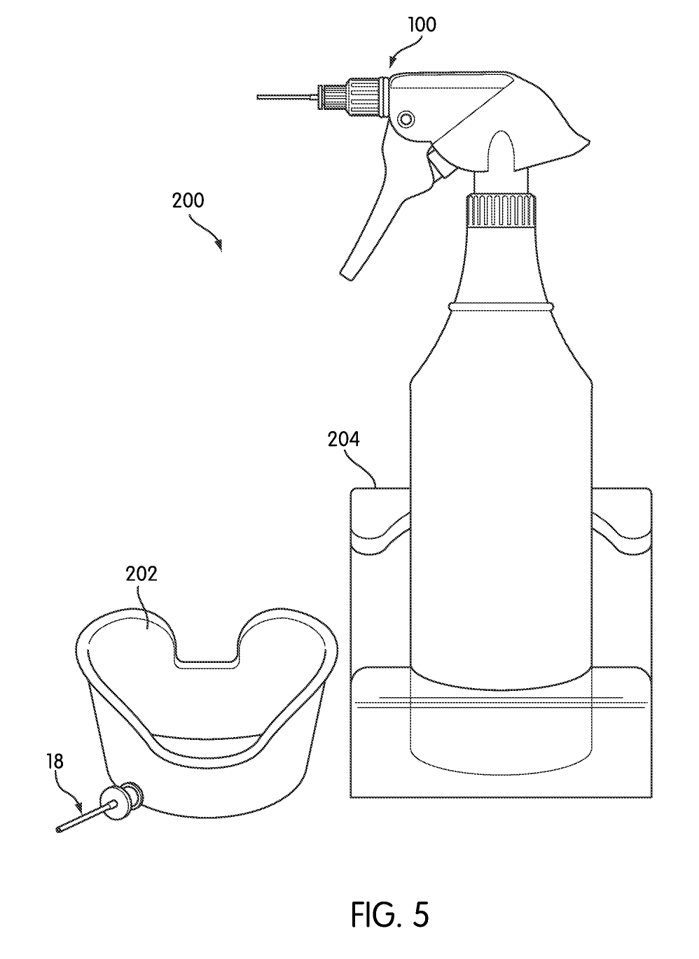

Irrigators 10, 100 according to embodiments of the invention may be sold as parts of a kit. FIG. 5 is a perspective view of a kit, generally indicated at 200, according to one embodiment of the invention. The kit 200 includes an irrigator, which in this case is irrigator 100, though any irrigator 10, 100 according to embodiments of the invention may be used. In addition to the irrigator 100, the kit 200 comprises one or more irrigating tips 18, and would generally include a plurality of irrigating tips 18, each made to be disposable. In addition, the kit 200 may include a basin 202, which may be shaped, contoured, and otherwise adapted to be placed just under the external portion of the ear to catch solution as it leaves the ear during and after the washing process. The basin 202 may be disposable or reusable and sterilizable by a standard process, such as by autoclaving.

The kit 200 may also include a bracket or holder 204 which is configured and adapted to hold the irrigator 10, 100 and may also include mounting hardware for mounting on a wall or in another such convenient location. In the illustrated embodiment, the bracket 204 is a straight-backed sheet of material that is bent at appropriate locations to define a back wall, a bottom, and a front wall. The ends of the bracket 204 are contoured to match the contours of the irrigator 10, 100, so as to provide a receptacle for the irrigator 10, 100. The bracket 204 may be made of a plastic, such as PLEXIGLAS®, a metal, or any other suitable material.

Any number of solutions may be used with irrigators 10, 100 according to embodiments of the invention. For example, a solution of 1 part 3% hydrogen peroxide solution and 4 parts water may be suitable. A minor portion (e.g., a few drops) of a surfactant or detergent, such as a conventional shampoo, may also be added. More dilute solutions, such as 1 part 3% hydrogen peroxide to 9 parts water, may be used. Other suitable solutions will be known to those of skill in the art.

The irrigators 10, 100 themselves may be disinfected by loading the reservoir 12 with a disinfectant solution and pumping it through the irrigator 10, 100 and by wiping the exterior with a disinfectant solution.

Although the invention has been described with respect to certain exemplary embodiments, the description is intended to be illuminating, rather than limiting. Modifications and changes may be made within the scope of the invention, which is defined by the appended claims.

What is claimed is:

1. An irrigation device, comprising:
a hand-held pumping device including a liquid reservoir and a pumping mechanism, the pumping mechanism having a liquid outlet in fluid communication with the liquid reservoir through the pumping mechanism, such that liquid drawn from the reservoir when the pumping mechanism is actuated is expelled from the liquid outlet; and
an irrigating tip directly connected to the liquid outlet, the irrigating tip including
a connector, and
an irrigation tube connected to the connector such that the connector places the irrigation tube in fluid communication with the outlet, the irrigation tube being made of a flexible material and having an inner diameter, an outer diameter, and a length suitable for insertion directly into the ear.

2. The irrigation device of claim 1, wherein the hand-held pumping device comprises a spray bottle with a manual trigger.

3. The irrigation device of claim 1, wherein the irrigation tube has a length of from about 0.5 inches to about 1 inch.

4. The irrigation device of claim 1, wherein the irrigation tube has an outer diameter of about ⅛ inch.

5. The irrigation device of claim 4, wherein the irrigation tube has an inner diameter of about 1/16 inch.

6. The irrigation device of claim 1, wherein the irrigation tube is constructed and arranged such that it will oscillate when liquid is projected through it by the pumping mechanism.

7. The irrigation device of claim 1, wherein the irrigating tip is connected to the outlet without any intervening conduit.

8. The irrigation device of claim 1, further comprising a sealing structure between the outlet and the connector.

9. The irrigation device of claim 8, wherein the sealing structure comprises an O-ring.

10. The irrigation device of claim 1, wherein the irrigating tip is removable and replaceable.

11. An irrigation device, comprising:
a hand-held pumping device including
a liquid reservoir,
a pumping mechanism, the pumping mechanism having a liquid outlet in fluid communication with the liquid reservoir through the pumping mechanism, such that liquid drawn from the reservoir when the pumping mechanism is actuated is expelled from the liquid outlet, and
an outlet connector member mounted on the liquid outlet; and
an irrigating tip directly connected to the liquid outlet, the irrigating tip including
a tip connector adapted to connect to the outlet connector member, and
an irrigation tube connected to the tip connector such that the tip connector places the irrigation tube in fluid communication with the outlet, the irrigation tube being made of a flexible material and having an inner diameter, an outer diameter, and a length suitable for insertion directly into the ear.

12. The irrigation device of claim 11, wherein the outlet connector member is generally tubular and changes diameter from a first diameter on a first end adapted to be mounted to the liquid outlet to a second diameter on a second end adapted to connect to the tip connector, the first diameter being greater than the second diameter.

13. The irrigation device of claim 11, wherein the hand-held pumping device comprises a spray bottle with a manual trigger.

14. The irrigation device of claim 11, wherein the irrigation tube has a length of from about 0.5 inches to about 1 inch.

15. An ear irrigation kit, comprising:
an irrigator, including a hand-held pumping device with a liquid reservoir and a pumping mechanism, the pumping mechanism having a liquid outlet in fluid communication with the liquid reservoir through the pumping mechanism, such that liquid drawn from the reservoir when the pumping mechanism is actuated is expelled from the liquid outlet, and a connector;
one or more irrigation tubes adapted to be connected to the connector such that the connector places the irrigation tube in fluid communication with the outlet, one or more irrigation tubes being made of a flexible material and having an inner diameter, an outer diameter, and a length suitable for insertion directly into the ear; and
a receptacle adapted to receive irrigation fluid flowing out of an ear during irrigation.

16. The ear irrigation kit of claim 15, further comprising a receptacle sized to receive and hold the irrigator.

17. A method of irrigating an ear, comprising:
placing the end of a short length of compliant tubing in the ear, the compliant tubing being a portion of an irrigating tip directly connected to an irrigation device, the irrigation device including a liquid reservoir and a pumping mechanism, the pumping mechanism having a liquid outlet in fluid communication with the liquid reservoir through the pumping mechanism, such that liquid drawn from the reservoir when the pumping mechanism is actuated is expelled from the liquid outlet and into the irrigating tip; and
actuating the pumping mechanism to direct irrigating fluid into the ear through the irrigating tip.

18. The method of claim 17, further comprising collecting irrigating fluid that escapes the ear in a container.

19. The method of claim 17, further comprising placing and operating the irrigation device with a single hand.
